(19) United States
(12) Patent Application Publication
(10) Pub. No.: US 2013/0030479 A1
(43) Pub. Date: Jan. 31, 2013

(54) BONE WEDGE

(71) Applicant: Markus Regauer, Oberaudorf (DE)

(72) Inventor: Markus Regauer, Oberaudorf (DE)

(73) Assignee: Ludwig-Maximilians-Universitat Munchen, Munchen (DE)

(21) Appl. No.: 13/640,089

(22) PCT Filed: May 14, 2010
(86) PCT No.: PCT/EP2010/056641

§ 371 Date: Oct. 9, 2012

(51) Int. Cl. A61B 17/84; A61B 17/88

(52) U.S. Cl. 606/324

(57) ABSTRACT

A bone wedge is provided for fixing tissue to bone. The bone wedge comprises an elongate body with a proximal end a distal end and a longitudinal axis (A) which extends between said proximal end and said distal end. The elongate body comprises at least two opposed sidewall portions between the proximal and distal ends, wherein the bone wedge is configured to be inserted into a wedge shaped cavity in the bone, said wedge shaped cavity having a smaller diameter at its portion proximal to the bone surface than at its distal portion, and wherein said two opposed sidewall portions are configured to clamp the tissue against the inner walls of said cavity.

The present invention generally relates to medical devices and procedures. More particularly, the present invention relates to appliances for use during certain orthopaedic surgical procedures, particularly those procedures involving replacing or securing a tissue like a tendon to a bone, as for example, to attach and maintain a ligament to a bone mass. In particular, the present invention relates to a bone wedge for attaching and fixing soft tissue to hard bone and to a method for attaching soft, tissue to hard bone.

### BACKGROUND OF THE INVENTION

In a practice of certain orthopaedic surgical procedures, particularly those involving attachment of a ligament or tendon to a bone mass, as for example, knee or elbow surgery, it has been a problem to effectively anchor that ligament to the bone moss for a long time.

Soft tissues, such as ligaments, tendons and muscles, are attached to a large portion of the human skeleton. In particular, many ligaments and tendons are attached to the bones which form joints, such as shoulder and knee joints. A variety of injuries and conditions require attachment or re-attachment of soft tissue to bone. For example, when otherwise healthy tissue has been torn away from a bone, surgery is often required to re-attach the tissue to the bone to allow healing and a natural re-attachment to occur. The tissue may be attached to the bone during open surgery, or during closed (e.g., arthroscopic) surgical procedures. Closed surgical procedures are preferred since they are less invasive and are less likely to cause patient trauma.

A number of devices and methods have been developed to attach soft tissue to bone. These include screws, staples, cement, suture anchors, and sutures alone. Some methods involve the use of a suture anchor to attach a suture to the bone, and tying the suture in a manner that holds the tissue in close proximity to the bone.

Proper attachment of soft tissue requires that it is placed in an anatomically correct position to promote optimal healing.

One conventional orthopaedic procedure for re-attaching soft tissue to bone is performed by initially drilling holes or tunnels at predetermined locations through a bone in the vicinity of a joint. Then, the surgeon approximates soft tissue to the surface of the bone using sutures threaded through these holes or tunnels. This method, although effective, is a time consuming procedure resulting in the generation of numerous bone tunnels. A known complication of drilling tunnels across bone is that nerves and other soft tissue structures may be injured by the drill bit or orthopaedic pin as it exits the far side of the bone. Also, it is anatomically very difficult to reach and/or secure a suture/wire that has been passed through a tunnel. When securing the suture or wire on the far side of the bone, nerves and soft tissues can become entrapped and damaged.

In order to overcome some of the problems associated with the use of conventional bone tunnel procedures, suture screws have been developed and are frequently used to attach soft tissue to bone. However, for fixing the screws to bone, a high torque is required such that the screws can break during attachment. Moreover, the sharp thread of the screw can easily damage the tissue and therefore weaken the tissue. On the other hand, a screw with a blunt thread might not provide a sufficient fixing in the bone, and proper application of a screw with a blunt thread can be quite demanding.

Moreover, suture anchors have been developed to overcome the disadvantages of suture screws. A suture anchor is an orthopaedic, medical device which is typically implanted into a bore drilled into a bone. Although less frequently, these devices have also been referred to as bone anchors. The bore hole does not extend through the bone. This type of bore hole is typically referred to as a “blind hole”. The bore hole is typically drilled through the outer cortex layer of the bone and into the inner cancellous layer. The suture anchor may be engaged in the bore hole by a variety of mechanisms including friction fit, barbs which are forced into the cancellous layer of bone, etc. Suture anchors are known to have many advantages including reduced bone trauma, simplified application procedures, and decreased likelihood of suture failure due to abrasion on bone. Also, such anchors may be used in repair of tendon tears by direct attachment of a tendon to a bone.

Suture anchors typically have at least one suture attached. This may be by means of a hole or opening for receiving the suture(s). At least one end and typically both ends of the suture strand extend out from the bore hole and are used to attach soft tissue. The suture anchors presently described in the art may be made from absorbable materials which absorb over time, or they may be made from various non-absorbable, biocompatible materials.

Although suture anchors for attaching soft tissue to bone are available for use by the orthopaedic surgeon, there is a constant need in this art for novel suture anchors having improved performance characteristics, as currently available suture anchors are associated with the following main disadvantages: usually a suture procedure is necessary for attaching tissue to bone, and the remaining suture material can cause local irritations; it is difficult to apply and to adjust proper tension to/of the tissue to be attached; immediate revision in case of inadequately applied tension is quite impossible; and it is difficult to provide sufficient contact between bone and tissue to be attached.

Accordingly, it is an object of the present invention to provide a mounting element and a method for mounting tissue to bone, which avoids the disadvantages of the prior art. In particular, it is an object of the invention to provide an improved system for anchoring soft tissue to bone and to prevent loosening of the anchor in the bone cavity. More particularly, it is an object of the present invention to provide a mounting element for mounting soft tissue like tendon to bone, which ensures that the tissue can be anchored and provides optimal purchase by minimal damage of the tissue.

The objects of the invention are achieved by the features of the independent claims. The dependent claims refer to further preferred embodiments of the invention.

### SUMMARY OF THE INVENTION

The present invention avoids the problems associated with conventional fixing screws or toggling anchors by providing a mounting element that secures a soft tissue within a bone cavity by minimizing damage of the tissue. Moreover, the method and the mounting element according to the present invention provide the advantage that tensile forces may be applied shortly after the attachment, as tensile forces are converted into pressure forces at the interface between bone and soft tissue to be attached to the bone.

The present invention provides a device or mounting element for fixing soft tissue, like a tendon, a sinew or a ligament, to hard bone. The device according to the present invention (in the following also called bone wedge or bone anchor or dowel) for fixing tissue to bone comprises an elongate body with a proximal end and a distal end, wherein said body extends between said proximal end and said distal end, preferably along a longitudinal (virtual) axis which extends between said proximal end and said distal end. The elongate body comprises at least two opposed sidewall portions between the proximal end and the distal end, wherein the sidewall portions are opposed with respect to the longitudinal axis. The bone wedge is configured to be inserted into a tapered cavity in the bone, wherein said tapered cavity has a smaller width/diameter at its portion proximal to the bone surface than at its distal portion. The tissue can be fixed to the bone by means of the two opposed sidewall portions which are adapted to clamp the tissue against the inner walls of said tapered cavity. The design of the bone wedge according to the present invention provides the advantage that at least the two opposed sidewalls—and not only one of the sidewalls—are configured to press the tissue against the inner walls of the cavity in the bone. Accordingly, substantially symmetric and/or substantially uniform distribution of forces to the cavity of the bone is achieved, and the contact area between bone and soft tissue is preferably increased.

Preferably an opening, in the following called tissue opening, is formed at the distal end of the bone wedge. This tissue opening is formed traverse to the longitudinal direction of the bone wedge. The opening is preferably a bore at the distal end. According to a further preferred embodiment, the opening is preferably offset from the centre of the longitudinal body. Such an opening provides the further advantage that the position between the tissue to be fixed and the bone wedge and preferably also the position relative to the cavity is substantially maintained even when strong forces are applied to the tissue.

The tissue opening is preferably cylindrically shaped. According to a further preferred embodiment, the opening is preferably elliptically or rectangularly shaped. It is further preferred that a substantially rectangularly shaped opening comprises chamfered edges and/or chamfered corners. The diameter of the opening or the cross-sectional area of the opening, respectively, is preferably adapted to the cross-section of the tissue and thereby to the cross-section of the wedge at its greatest diameter. In other words, in case the tissue is a tendon, the cross-sectional area of the tissue opening lisq to be at least equal to or larger than the cross-sectional area of the tendon. Preferably the cross-sectional area of the tissue opening has to be at least equal to or larger than the cross-sectional area of the wedge at its greatest diameter.

The bone wedge according to the present invention is preferably not cylindrically shaped such that the cross-section perpendicular to the longitudinal axis varies between the proximal and the distal end. According to a preferred embodiment, the cross-section of the wedge—or in other words the diameter of the wedge—comprises the maximum value somewhere between the proximal end and the distal end. Still in other words, the wedge is preferably egg-shaped, lemon-shaped or diamond-shaped, when viewed from a side perpendicular to the opposed side walls. Furthermore, the cross-section is preferably substantially rectangularly shaped at the proximal and/or the distal end and preferably substantially cylindrically shaped in a middle portion, wherein the middle portion ranges preferably ±30% of the longitudinal dimension of the wedge from the geometrical mean of the wedge.

The maximum cross-section of the elongate body perpendicular to the longitudinal axis is preferably located in the middle portion. According to a further preferred embodiment the maximum cross-section (also referred to as “maximum diameter”) is preferably located within the proximal third of the elongate body. It may be further advantageously if the elongate body comprises at least one tooth, preferably a plurality of teeth at the position of the maximum cross-section. The “maximum diameter” of the bone wedge is preferably only slightly smaller that the smallest diameter of the cavity in the bone receiving the bone wedge together with the soft tissue. It is farther preferred that the sum of the “maximum diameter” of the bone wedge and twice the diameter of a filament is smaller that the smallest diameter of the cavity in the bone.

According to a further preferred embodiment, the cross-sectional area of the opening is preferably equal to or larger than the maximum cross-sectional area of the elongate body perpendicular to the longitudinal axis. This provides the advantage that the diameter of the bore hole in the bone can be designed as small as possible and that passing of the tissue through the tissue opening can be performed safely and without use of great force.

The bone wedge according to a further preferred embodiment comprises at least two or more teeth. The term “tooth” or “teeth” refers to projections from the surface of the body which increase(s) the friction between the bone wedge and the tissue. In other words, the teeth of the bone wedge or bone dowel are preferably designed as ridges, corrugations, riffles, spikes or pins protruding outwards from the surface of the body.

The bone wedge according to the present invention is preferably formed out of titanium, although other suitable materials may also be used. For instance, the bone wedge according to the present invention may be formed out of plastic or metal, like titanium, or a bio-absorbable material or any other biocompatible material. Preferably both, the body and the teeth (spikes or pins) are made up of plastic or metal or any other bio-absorbable or biocompatible material. For instance, the bone wedge according to the present invention may be formed by extrusion, by injection moulding, or by spark or wire eroding.

According to another preferred embodiment, the surfaces or part of the surfaces of the sidewalk may be roughened. According to a further embodiment, the teeth may be designed as simple hooks or barbs. It is further preferred that each of the two opposed sidewalls of the bone wedge comprises at least one tooth, preferably a plurality of teeth.

The teeth, which increase the friction between the bone wedge and the tissue may be provided in any kind of order or arrangement. According to a preferred embodiment, the teeth are formed on the two opposed sidewalls. According to a further preferred embodiment, at least one of the teeth is preferably inclined or slanted with regard to a plane perpendicular to the local surface outline of the wedge. The angle of inclination preferably ranges between 1° and 89°, more preferably between 30° and 60°.

For instance, a tooth which is inclined in the direction to the proximal end, e.g., the peak of the tooth is directed towards the proximal end of the bone wedge, increases the friction between the bone wedge and the tissue when a pulling force is acting on the tissue (a force at the tissue being directed away from the bone surface).

According to a further preferred embodiment, at least one of the teeth of each of the two opposed sidewalls is inclined/slanted towards an opposite direction. For instance the tooth or teeth on a first sidewall are slanted towards the proximal end of the body, whereas the tooth or teeth on a second sidewall are slanted towards the distal end of the body, or vice versa. In other words, the slanting direction of the teeth on the two opposed sidewalls is contrary.

According to a further preferred aspect of the present invention, the teeth of a first sidewall of the two opposed sidewalls are shifted along the longitudinal axis with respect to the teeth of the other sidewall of the two opposed sidewalls. In other words, there exists an offset in the longitudinal direction between corresponding teeth on the two opposed sidewalls.

According to a preferred embodiment, the teeth on a sidewall are arranged in a matrix form, i.e., in columns and rows. Preferably both opposed sidewalls have such a matrix arrangement.

According to still a further preferred embodiment, the teeth of a sidewall are shifted along the longitudinal axis with respect to an adjacent tooth on said sidewall. In other words the teeth are staggered on the sidewall. Still in other words, the teeth are arranged in a matrix form, but the rows and/or the columns are shifted or staggered with respect to the adjacent row/column.

According to a further preferred embodiment of the present invention, a longitudinal groove (a groove oriented parallel to the longitudinal axis of the bone wedge) may be provided between the proximal end and the opening of the wedge. Such a groove is preferably provided on at least one of the sidewall portions and preferably on both of the two sidewall portions. The groove comprises preferably a depth/diameter to accommodate the guiding filament (Ethibond® or Ethibond® 3-0, for example) inside the groove. Preferably, the depth and/or the diameter of the groove is larger than the diameter (thickness) of the filament such that the entire filament may be located inside the groove. This provides the advantage that the bone wedge together with the filament—which is guided from the proximal end at the first sidewall portion to the opening and back to the proximal end along the second side wall portion—can be inserted into a bore (cavity) in the bone, wherein the diameter of the bore is equal to or only slightly larger than the maximum diameter of the bone wedge.

According to a further preferred embodiment, the depth and/or the diameter of the groove is smaller than the diameter (thickness) of the corresponding filament. Accordingly only a portion of the filament (not the entire filament) is located inside the groove. Such a design, however, still provides the advantage that the filament is definitely guided from the proximal end of the bone wedge to the opening along a first sidewall portion and back to the proximal end along the second sidewall portion during insertion of the bone wedge into the bore and the cavity in the bone.

According to still another preferred embodiment, the teeth are arranged along a right-handed or left-handed helical curve/thread.

In order to simplify the insertion of the bone wedge according to the present invention into the cavity of the bone, an insertion tool may be used which can be detachably coupled to the bone wedge. The insertion tool preferably comprises a shaft and preferably comprises a handle. The shaft of the insertion tool is preferably insertable into a bore or mounting opening of the bone wedge. Such a bore is preferably located at the proximal end of the bone wedge and is preferably aligned along with the longitudinal axis. According to another preferred embodiment, the shaft and the bone wedge may be coupled via a bayonet fitting. Still according to another preferred embodiment, the bore may comprise a female thread such that the shaft may be screwed into the threaded bore. In general the mounting opening need not be cylindrical in shape. For instance, the mounting opening may be shaped as to prevent rotation of the bone wedge during insertion.

The present invention also relates to a kit which comprises tools useful for the fixing of a tissue to a bone according to the present invention. In particular, the kit according to the present invention preferably comprises a bone wedge according to the present invention and an insertion tool according to the present invention. The kit may also comprise a filament for extending a free end of the tissue to be fixed for handling purposes only.

The present invention also relates to a method for fixing tissue to bone by means of a bone wedge according to the present invention. The method comprises preferably the following steps:

a) Firstly a tapered cavity is formed in the bone, wherein the cavity has a smaller diameter at its portion proximal to the bone surface than at its distal portion. The tapered cavity may be drilled with a conventional drill or with a special tool, which may also be a part of the above discussed kit. The tapered cavity may also be created by drilling just the cortex and impacting the cancellous bone by use of a special tool.

b) Next, the bone wedge and the insertion tool are joined; e.g. the shaft of the insertion tool is releasably connected to the (mounting opening at the) proximal end of the bone wedge.

c) A free end of the tissue to be fixed is extended with a (guiding) filament which has preferably a smaller thickness than the tissue. The guiding filament is merely used and useful for the next steps d) and f). In other words, the guiding filament is merely used to assist the surgeon for inserting the tissue through the opening in the bone wedge (see steps d) and f) below).

d) The filament is inserted into the opening of the bone wedge.

e) The bone wedge is inserted together with said filament into the cavity such that the filament is located along a path from the outside of the cavity into the cavity and there from the proximal end of the body along the teeth of a first sidewall through the opening at the distal end and back to the proximal end of the wedge along the teeth of the opposed sidewall and out from the cavity.

f) Finally, the surgeon is pulling at the filament such that the attached tissue will follow the path of the filament from the outside into the cavity, around the wedge and back to the outside of the cavity.

It should be noted that the above steps a) to f) may be performed in a slightly different order. For instance it would also be possible to connect the insertion tool with the bone wedge after step c) or d).

It is farther preferred that the tapered cavity in the bone comprises a depth which is between 1.5 and 2 times the depth of the longitudinal extension of the bone wedge.

The bone wedge and the method according to the present invention may be used for a variety of injuries and conditions which require attachment or re-attachment of a soft tissue to bone. For example, when otherwise healthy tissue has been torn away from a bone, the method with the bone wedge according to the present invention may be used for reattaching the tissue to the bone to allow healing and a natural reattachment to occur. Furthermore, if the tissue which has been torn away is too short for a reattachment with a bone wedge according to the present invention, the tissue may be extended by fixing an additional tissue to the torn away end of the tissue. The tissue for extending the natural tissue may be any kind of tissue, like natural tissue, artificial tissue which may be absorbable, partly absorbable, slowly absorbable or non-absorbable. Possible suitable materials are known in the art, e.g. FiberTape™. For instance, the artificial tissue may be formed as an ultra-high strength tape using long chain polyethylene structures. The artificial tissue preferably comprises three regions, a distal region, a mid region and a proximal region. The diameters and/or strength of the distal and proximal regions are preferably smaller than the diameter and/or strength of the mid region. Such designed artificial tissues provide the advantage that the proximal region may be easily fixed to the torn away end of the tissue and the distal end of the artificial tissue may be used as a guiding filament (see e.g. the above steps b), d) and f). In other words, the distal end is used to assist the surgeon for inserting the tissue through the opening in the bone wedge (see steps d) and f) above). On the other hand, the mid region with a larger diameter is preferably used for engaging with the bone wedge in the cavity of the bone. Accordingly, the larger diameter mid region provides a secure fixation of the tissue within the bone cavity. Moreover, such a construction may also allow that at least a part of the torn away tissue is inside the cavity when the tissue is fixed to the bone. Accordingly, the bone wedge preferably engages with the natural tissue and the artificial tissue. The fact that at least a part of the natural torn away tissue is reinserted into the bone cavity may positively enhance the healing process. In other words, since a direct contact between the natural tissue and cancellous bone is possible, the healing process is enhanced over known prior art methods.

Further features of the invention, its nature and various advantages, will be more apparent from the accompanying drawings and the following detailed description of the drawings and the preferred embodiments.

### BRIEF DESCRIPTION OF THE DRAWING

FIG. 1a is a side view of a bone wedge according to a preferred embodiment of the invention;

FIG. 1b is a front view of the bone wedge of FIG. 1a;

FIG. 2 is a cross-sectional view of a bone wedge inside a tapered cavity during the inserting step;

FIGS. 3a-c are views from the front (a), from the side (b) and from the back (c) of an embodiment of the bone wedge according to the present invention;

FIG. 3d shows cross-sectional views made from the top at a proximal end, a middle portion and a distal end of the bone wedge of FIGS. 3a-c;

FIGS. 3e-i are front views of different embodiments of bone wedges according to the present invention showing different designs of the teeth;

FIG. 4a shows a side cross-sectional view and a cross-sectional view perpendicular to the axis of the drilling hole of a conventional bore in a bone;

FIG. 4b shows a side cross-sectional view and a cross-sectional view perpendicular to the axis of a cavity in a bone according to the present invention;

FIGS. 5a-5k show individual steps of the method for fixing a tissue to bone according to the present invention;

FIGS. 6a-6c show a method of extending a natural torn away tissue with an artificial tissue; and

FIG. 7 shows an extended torn away tissue fixed to bone according to the present invention.

### DETAILED DESCRIPTION OF THE DRAWINGS

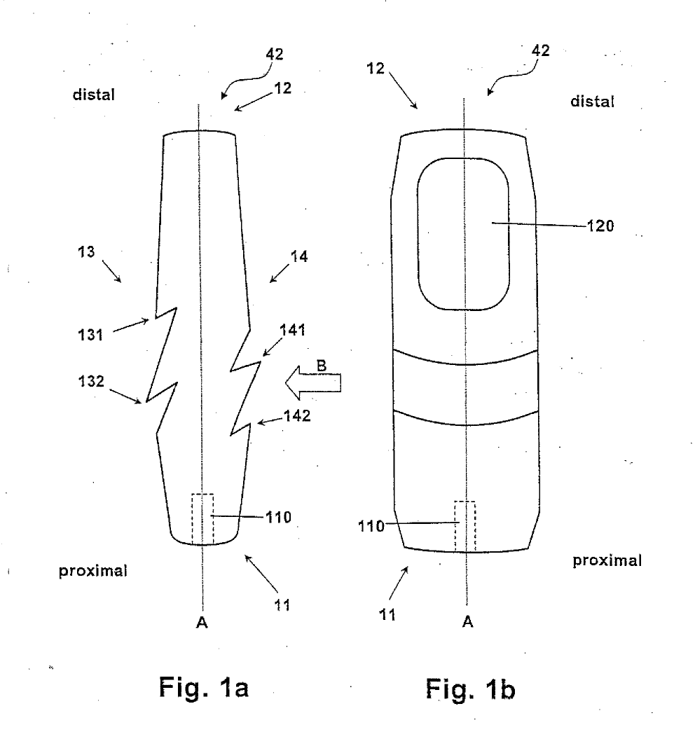

Referring to FIGS. 1a and 1b, an exemplary bone wedge or bone anchor 42 of the present invention is shown having an elongate body extending between a first (leading) end 11 and a second (trailing) end 12 along its longitudinal axis A.

In particular, FIG. 1a shows a side view of the bone wedge 42 according to the present invention with a first side wall 14 on the right side (see arrow B) and a second side wail 13 on the left side opposite to the first side wall 13. The side walls 13 and 14 are located between the leading (proximal) end 11 and the trailing (distal) end 12 of the bone wedge 42.

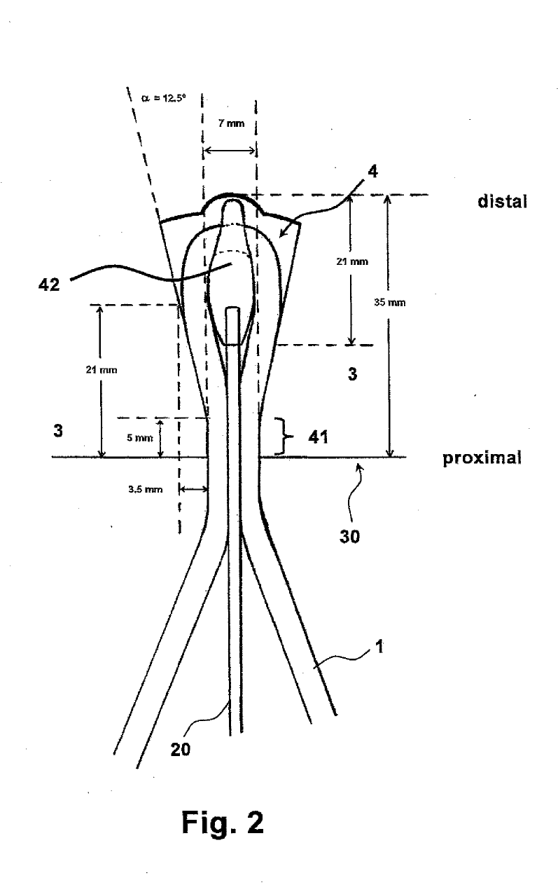

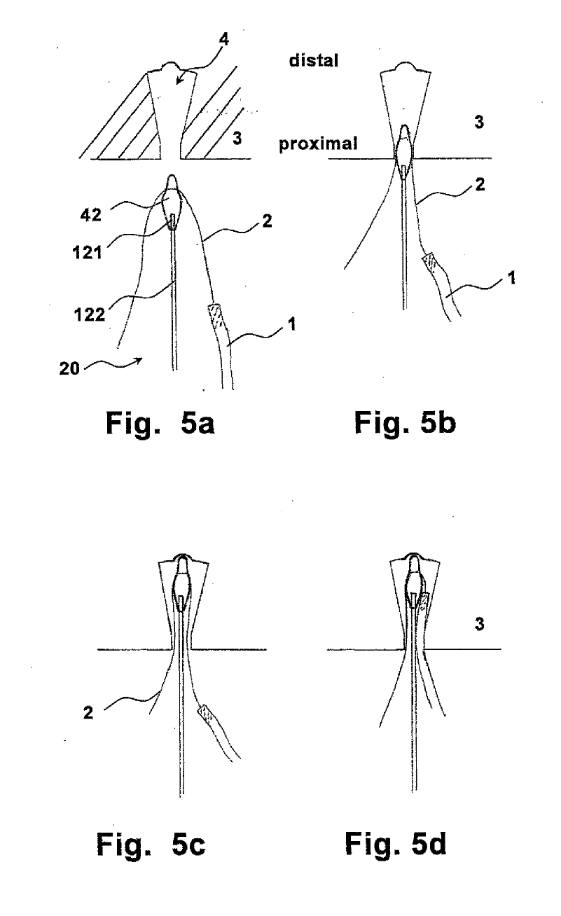

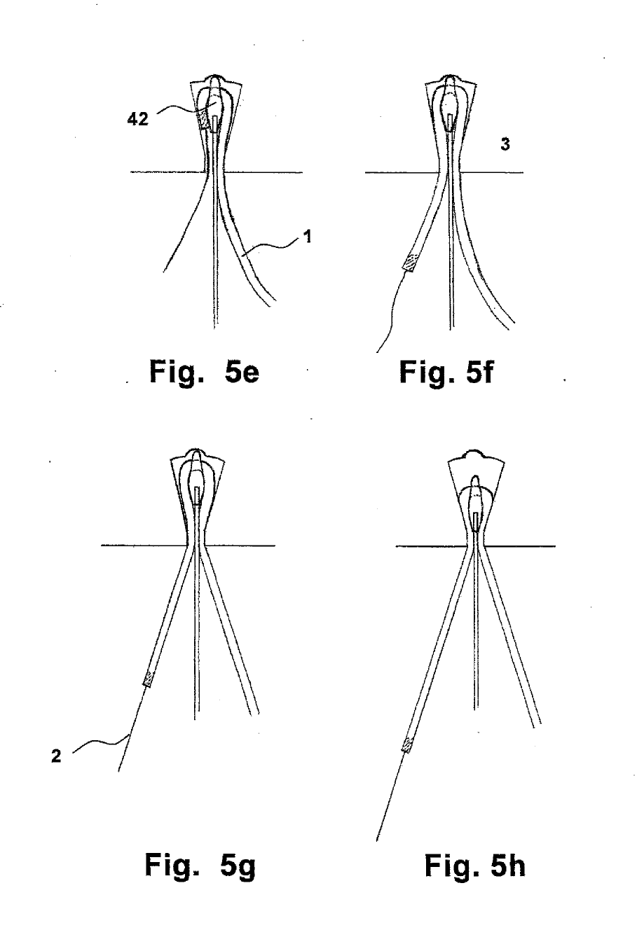

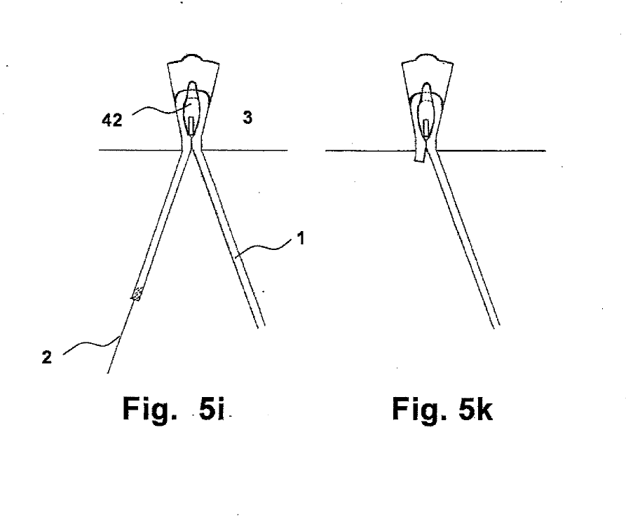

FIG. 1b shows a front view of the bone wedge 42, in particular, a front view of the first side wall 14 (see arrow B). The bone wedge 42 according to the present invention is particularly designed and configured to be inserted into a cavity 4 (see e.g. FIGS. 2 and 5a) which is formed in a bone 3, wherein the cavity 4 in the bone 3 is not a conventional cylindrical bore but preferably a tapered or wedge-shaped cavity 4.

The wedge-shaped cavity 4 has a smaller diameter at its proximal portion, i.e., the portion proximal to the bone surface, than at its distal portion. The wedge-shaped or tapered cavity 4 is, for instance, depicted in FIGS. 2, 4, and 5a to 5k. In particular, FIG. 2 shows the dimensions of an exemplary embodiment which will be discussed in more detail below.

The two opposed side wall portions 14 and 13 are particularly configured to clamp a tissue 1 to be fixed to the bone 3 against the inner wall(s) of the tapered cavity 4. In particular, it should be noted that both side walls 13 and 14 of the bone wedge 42 are preferably configured to clamp the tissue 1 against the inner wall of the tapered cavity 4. In other words, the bone wedge 42 according to the present invention is specifically designed such that a soft tissue 1 is located around the bone wedge 42 along the two opposed side walls 14 and 13. In case an external pulling force is applied to the tissue 1, both side walls 14 and 13 will be pressed against the tissue 1, wherein the tissue is pressed against the inner wall of the cavity 4. In other words, the bone wedge 42 fixes the tissue 1 by clamping. Preferably, the tissue 1 is located around the bone wedge 42 along a path from the proximal end 11 of the first side wall 14 to the distal end 12 of the first side wall 14, through a tissue opening 120 and back from the distal end 12 of the second side wall 13 to the proximal end 11 of the second side wall 13. For instance, FIG. 2 shows a bone wedge 42 located within the cavity 4 with a soft tissue 1 located around the bone wedge 42. In order to maintain the alignment between the tissue 1, the bone wedge 42 and the tapered cavity 4, the tissue opening 120 is preferably formed at the distal end 12 of the bone wedge 42. The tissue opening 120 next to the distal end 12 of the bone wedge 42 may comprise any desired form, e.g., a round or elliptical cross-section, wherein the cross-section of the tissue opening 120 is preferably equal to or larger than the diameter of the soft tissue 1 to be fixed. Moreover, the cross-sectional area of the (tissue) opening 120 is preferably equal to or larger than the maximum cross-sectional area of the elongated body of the bone wedge 42, which provides further advantages which will be described in further detail with regard to FIGS. 5a to 5h.

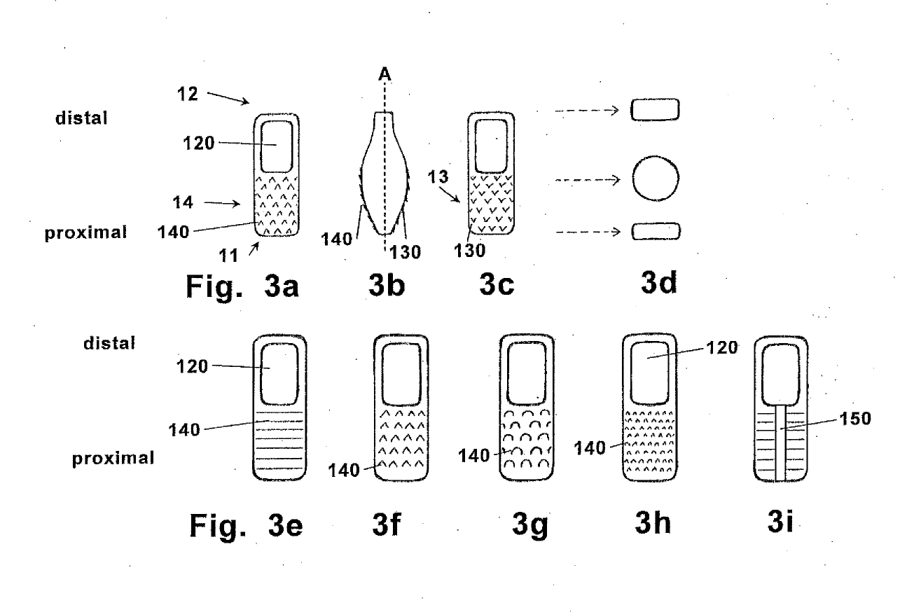

Moreover, in order to enhance the friction between the bone wedge 42 and the tissue 1 (to enhance the clamping capability of the bone wedge 42), there are at least two or more teeth formed at the first side wall 14 and/or the second side wall 13. As mentioned above, the term “tooth” or “teeth” refers to any kind of projections from the surface of the body which help to increase the friction between the bone wedge 42 and the tissue 1. FIGS. 1a and 1b show the teeth formed as ridges at the two side walls 13 and 14. In particular, the embodiment as shown in FIGS. 1a and 1b shows two teeth (ridges) 141 and 142 at the first side wall 14 and two teeth (ridges) 131 and 132 at the second surface 13 opposite to the first surface 14. As can be seen in FIG. 1a, the teeth 141 and 142 of the first side wall 14 are slanted backward, i.e., the teeth are inclined such that the tip projects towards the distal end 12, whereas the teeth 131 and 132 are slanted towards the proximal end 11 of the bone wedge 42. In other words, the teeth 141 and 142 of the first side wall 14 are inclined opposite to the teeth 131 and 132 of the opposed side wall 13. Still in other words, the teeth 131 and 132 are oriented contrariwise. Further preferred embodiments are shown in FIGS. 3a-3h.

Furthermore, a mounting opening 110 is provided at the proximal end 11 of the bone wedge 42. This mounting opening 110 is preferably sized to receive a mounting end 121 of an insertion tool 20 as shown in FIG. 2 and FIGS. 5a to 5h. The insertion tool 20 having a mounting end 121 is comprised of an elongated shaft 122. In use, the mounting end 121 of the insertion tool 20 is received within the mounting opening 110 of the body of the bone wedge 42.

FIGS. 3a and 3e to 3h show front views of a first side wall 14 of different embodiments of a bone wedge 42 according to the present invention. In particular the shown first side wall 14 corresponds to the first side wall 14 as shown in FIG. 1a (see arrow B), i.e., the FIGS. 3a and 3e to 3h show different embodiments of FIG. 1b. For instance, FIG. 3a shows an embodiment with a plurality of staggered arranged teeth 140 at the proximal part of the bone wedge 42 and a largely sized tissue opening 120 located at the distal end 12 of the bone wedge 42. In particular, the teeth 140 are formed similar to the teeth of a rasp or grater. It should be noted that the teeth 140 are directed towards the distal end 12, i.e., the teeth 140 are slanted in the direction of the distal end 12. FIGS. 3f and 3h show similar embodiments, whereas the teeth 140 are aligned in a matrix manner, i.e., in a table with parallel columns and parallel rows, wherein each cell of the table comprises a tooth. In other words, the staggered array as shown in FIG. 3a may be characterized as a table with parallel columns but with staggered rows. Still in other words, the staggered array of FIG. 3a may be characterized as a table with parallel TOWS and parallel columns, but with empty cells. Moreover, FIG. 3g shows an embodiment which is similar to the embodiments of FIGS. 3f and 3h, whereas the teeth 140 are chamfered, which may further reduce the risk of tissue rupture. The embodiment of FIG. 3h is similar to the embodiment of FIG. 3f, whereas the number of teeth 140 is higher in FIG. 3h, but the size of the teeth 140 with regard to FIG. 3f is reduced. Finally, FIG. 3e shows the teeth 140 in the form of ridges, similar to the surface of a file, where the surface is mostly covered with a series of sharp, parallel ridges or teeth. In other words, the embodiment shown in FIG. 3h is similar to the embodiment shown in FIG. 1b.

FIG. 3c shows a front view of the second side wall 13, i.e., the surface which is opposed to the surface 14. As can be seen, the teeth 130 are directed towards the proximal end 11, i.e., the teeth 130 are slanted towards the proximal end 11. In other words, the teeth 130 of the second sidewall 13 are orientated opposite to the teeth 140 of the opposed first side wall 14. The opposite orientation of the teeth may be also seen from FIG. 3b, which is a side view of the bone wedge of FIGS. 3a and 3c (see also similar side view of FIG. 1a). Moreover, as can be seen from FIG. 3b, the bone wedge 42 according to the present invention is preferably not cylindrically shaped along its longitudinal axis A, but comprises at its proximal end 11 and at its distal end 12 a cross-section (traverse to the longitudinal axis A) which is smaller than the cross-section in a mid portion (see FIG. 3d). In other words, the exemplary depicted bone wedge 42 is substantially shaped like a lemon. This design is furthermore obvious from the illustration of FIG. 3d which shows three cross-sections of the bone wedge 42 according to the present invention which are made at the proximal end 11, a mid portion and the distal end 12. Furthermore, it should be noted that the cross-sections at the proximal end 11 and the distal end 12 are substantially rectangularly formed, whereas the cross-section taken at the middle portion of the bone wedge 42 is substantially circularly formed.

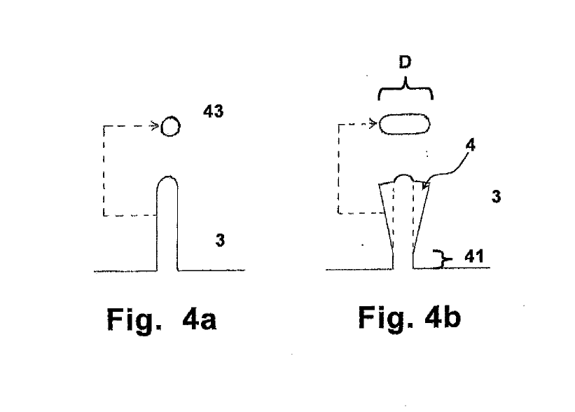

The bone wedge 42 according to the present invention is preferably designed to be inserted into a tapered or wedge-shaped cavity 4 in a bone 3. FIGS. 4a and 4b show, how such a tapered cavity 4 may be formed in a bone 3. In a first step, the surgeon drills a substantially cylindrical hole in the bone 3. FIG. 4a shows in the below drawing a side view of the bore, whereas the upper drawing shows the cylindrical cross-section 43 of the bore. In the next step, the surgeon creates a cavity 4 with a tapered form. Such a tapered cavity 4 may be formed by inclining the drill with regard to the bone surface, e.g., the “diameter” D of the “bore” is enlarged at the portion of the bore which is inside the bone 3. Such a tapered cavity 4 may, however, be formed by a special tool, which may be a part of a kit according to a preferred embodiment according to the present invention. It should be further noted that this particular embodiment of the cavity 4 comprises a substantially cylindrically shaped front part 41 proximal to the surface of the bone 3 with a diameter substantially equal to the diameter of the initial bore (depicted as broken line in FIG. 4b).

FIG. 2 shows the dimension of a cavity 4 and a corresponding bone wedge 42 according to an exemplary embodiment of the present invention in more detail. As obvious for a person skilled in the art, the dimensions are not to be interpreted as limiting, since the dimensions are dependent on the particular shape of the bone 3, the tissue 1 and the bone wedge 42. As can be seen, a bone wedge 42 is located inside the cavity 4 together with the soft tissue 1 placed armed the bone wedge 42. The position of the bone wedge 42 with regard to the cavity corresponds to the implantation procedure step as depicted in FIG. 5g, wherein the whole implantation procedure will be discussed in more detail below.

The exemplary embodiment as shown in FIG. 2 shows a cavity 4 with a total depth of 35 mm, wherein the cavity 4 preferably comprises a substantially circular bore portion 41 with a length of 5 mm proximal to the bone surface. The basic diameter of the bore is 7 mm. The angle of the tapered cavity 4, i.e., the angle of the inner surface of the cavity 4 with regard to a centre axis of the bore is α=12.5°. The longitudinal length of the elongated body of the bone wedge 42 is 21 mm. Generally, it is preferred that the longitudinal length of the bone wedge is smaller than the length of the tapered part of the cavity 4. In particular, in the depicted embodiment, the tapered part of the cavity 4 is the total depth minus the cylindrically shaped insertion bore portion 41 proximal to the bone surface: 35 mm−5 mm=30 mm>21 mm. The maximum diameter (taken traverse to the longitudinal axis A) of the bone wedge 42 is preferably slightly smaller than the basic diameter of the bore (basic bore diameter 7 mm; maximum diameter of bone wedge 42<7 mm) In the depicted embodiment of FIG. 2 the basic diameter of the bore is 7 mm wherein the diameter of the bone wedge is slightly smaller than said 7 mm. The thickness of the tendon 1 to be fixed is approximately 3.5 mm.

With regard to FIGS. 5a to 5k, the method according to the present invention for fixing soft tissue, e.g., a tendon 1 to bone 3 by means of a bone wedge 42 according to the present invention will be discussed in more detail. It should be noted that a single tendon 1 or a plurality of tendons may be fixed to bone 3 by the present method. Moreover, the fixation method according to the present invention may be applied to one free end of a tendon 1 (the other end of the tendon is still naturally adhered to the bone) or to two free ends, e.g., in case a tendon graft 1 is fixed to bone 3 (e.g., in the case of cruciate ligament replacement).

In a first step, a tapered cavity 4 is formed in a bone 3, e.g., by means of a conventional drill or by means of a special tool as shown in FIGS. 4a and 4b. The bone wedge 42 to be inserted into said cavity 4 is connected to the shaft 122 of the insertion tool 20 at the proximal end 11. For instance, the shaft 122 is inserted into the mounting bore 110. A free end of the tendon 1 to be fixed to bone 3 is extended by a (guiding) filament 2. Preferably, the filament 2 has a diameter which is substantially smaller than the thickness of the tendon 1 to be fixed. The filament 2 is merely used for inserting the tendon 1 into the cavity 4 and around the bone fixing wedge 42. Preferably, the filament 2 does not remain within the cavity 4 when the tendon 1 is fixed to the bone 3, as shown in FIGS. 5d to 5k. In other words, the filament 2 is merely used as a “guiding wire”, whereas instead of a relatively rigid wire a flexible filament is preferred. The filament 2 is guided along the first side wall 14 from the proximal end 11 to the distal end 12 of the bone wedge 42, through the opening 120 and back from the distal end 12 to the proximal end 11 of the bone wedge 42.

In a next step (FIG. 5b) the bone wedge 42 according to the present invention is inserted together with the filament 2 through the cylindrical bore of the cavity 4 proximal to the bone surface, wherein the diameter of the bore is preferably slightly larger than the maximum diameter of the bone wedge 42 plus twice the diameter (thickness) of the filament 2; otherwise an insertion of the bone wedge 42 together with the filament 2 would be impossible. According to another preferred embodiment of the present invention, a groove 150 may be provided between the proximal end 11 and the opening 120 of the wedge 42 (see e.g. FIG. 3i). Such a groove 150 is preferably provided on at least one of the sidewall portions 13 and 14 and preferably on both of the two sidewall portions 13 and 14. Moreover, the depth and/or the diameter of the groove 150 is provided in such a manner that the filament 2 is located substantially inside said groove 150. Preferably, the depth and/or the diameter of the groove 150 is larger that the diameter (thickness) of the filament 2 such that the entire filament 2 can be located inside the groove 150. This provides the advantage that the diameter of the bone wedge 42 with the “two” guiding filaments 2 inside the grooves 150 is not enlarged such that the smallest diameter 43 of the bore is equal to or only slightly larger than the maximum diameter of the bone wedge 42.

According to a further preferred embodiment, the depth and/or the diameter of the groove 150 may be smaller than the diameter (thickness) of the corresponding filaments 2. Accordingly only a portion of the filament 2 (not the entire filament 2) is located inside the groove 150. Such a design has still the advantage that the filament 2 is securely guided from the proximal end 11 of the wedge 42 to the opening 120 and back to the proximal end 11 during insertion of the bone wedge 42 into the bore cavity 4. The groove 150 may be provided with any embodiment of the present invention, i.e., the groove 150 is not limited to the specific embodiment of FIG. 3i.

The bone wedge 42 with the filament 2 guided around the bone wedge 42 is further advanced into the cavity 4, preferably until the bone wedge 42 abuts at the distal end of the cavity 4 (FIG. 5c).

In a next step, the surgeon pulls at the filament 2 such that the “free” end of the tendon 1, which is connected to the filament 2, is inserted into the bore of the cavity 4 (FIG. 5d).

Accordingly, the tendon 1 is guided along the first side wall 14 from the proximal end 11 to the distal end 12 of the bone wedge, through the opening 120 at the distal end 12 of the bone wedge 42. It should be noted that the bone wedge 42 preferably comprises teeth 140 which are directed (slanted) towards the distal end 12 of the bone wedge 42, such that the guiding of the tendon 1 along a first side wall 14 within the cavity 4 is possible. In other words, the slanting direction of the teeth 140 and the guiding direction of the tendon 1 are the same. By further pulling at the filament 2, the tendon 1 is guided back along the second side wall 13 of the bone wedge 42, i.e., guided from the distal end 12 towards the proximal end 11 along the second side wall 13 of the bone wedge 42 (FIG. 5e). The teeth 130 of the second side wall 13 are preferably slanted in the guiding direction of the tendon 1, i.e., from the distal end 12 towards the proximal end 11, such that a movement of the tendon 1 around the bone wedge 42 is not unduly hindered. It should be noted that guiding of the tendon 1 around the bone wedge 42 according to the present invention is preferably enhanced if the bone wedge 42 is located at its deepest position in the cavity 4, i.e., there is sufficient space around the bone wedge 42 within the cavity 4 for allowing such a guidance.

By further pulling at the filament 2, the tendon 1 is finally guided out of the cavity 4 through the bore portion proximal to the bone surface (FIG. 5P.

In such a position, the surgeon may adjust the effective length of the tendon 1 by pulling at the free end (left part of tendon 1 in FIG. 5g) and/or a fixed part of the tendon (right part of tendon 1 in FIG. 5g) while simultaneously pushing the bone wedge 42 into the cavity 4 by pushing the insertion tool 20 against the distal end of the cavity 4. Accordingly, the surgeon is able to control the position of the bone wedge 42 and the tendon 1 within the cavity 4 (FIG. 5g).

After adjusting the effective length of the tendon 1, the surgeon preferably pulls at one or both parts of the tendon 1 which extends from the bone cavity. Preferably, the surgeon pulls at the one or both parts of the tendon 1 while simultaneously decreasing the pressure on the insertion tool 20, outwardly, such that the tendon 1 is clamped by means of the two side walls 13 and 14 of the bone wedge 42 inside the cavity 4 (FIG. 5h).

Finally, the insertion tool 20 is disconnected (FIG. 5i) and the free end of the tissue 1 is shortened (FIG. 5k).

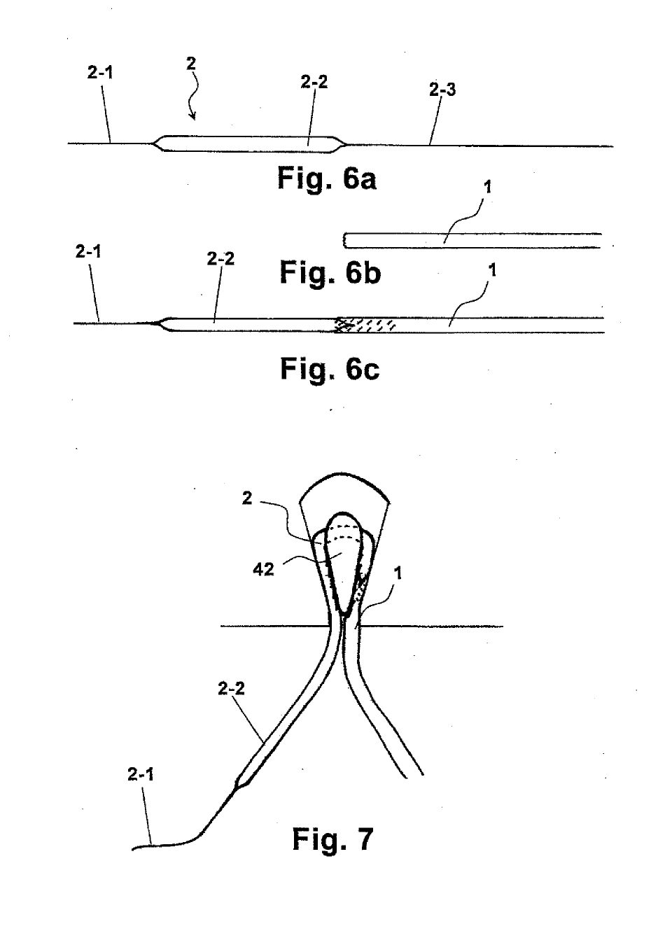

FIGS. 6 to 7 show a further method according to the present invention. In particular, if a severed natural tendon 1 is too short for a reattachment with a bone wedge according to the present invention, the tendon 1 may be extended by fixing an artificial tendon 2 to the natural tendon 1. FIG. 6a shows an artificial extension tendon 2 with a distal section 2-1, a mid section 2-2 and a proximal section 2-3. The diameter or thickness of the mid section 2-2 is larger than the diameters of the distal and/or proximal section.

FIG. 6b shows the free end of the severed natural tendon 1. FIG. 6c shows the artificial tendon 2 which is fixed by means of the proximal section 2-3 to the free end of the severed natural tendon 1. The proximal section 2-3 with a substantially smaller diameter filament may be fixed to the tendon by knotting or suturing techniques.

The distal section 2-1 of the artificial tendon may be used in the same manner as the filament 2 in FIGS. 5a to 5i.

As can be seen in FIG. 7, the end of the natural tissue 1 and the mid section of the artificial tissue 2 are both located inside the cavity and are in frictional engagement with the bone wedge 42. The larger diameter mid section provides a secure fixation of the tendon in the cavity due to the larger diameter of the mid section. The mid section 2-2 of the artificial tendon 2 has preferably the same diameter as the severed tendon 1 which provides the further advantage that the engagement forces between the bone wedge and the tendons 1 and 2 are uniformly distributed. Moreover, the fact that at least a part of the severed tendon 1 is reinserted into the bone cavity may positively enhance the healing process.

While the invention has been illustrated and described in detail in the drawings and foregoing description, such illustration and description are to be considered illustrative or exemplary and not restrictive; the invention is not limited to the disclosed embodiments. Variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

1. Bone wedge for fixing tissue to bone, comprising:
an elongate body with a proximal end, a distal end and a longitudinal axis (A) which extends between said proximal end and said distal end;
the elongate body comprises at least two opposed sidewall portions between the proximal and distal ends;
characterized in that
the bone wedge is configured to be inserted into a tapered cavity in the bone, said cavity having a smaller diameter at its portion proximal to the bone surface than at its distal portion,
wherein said two opposed sidewall portions are configured to clamp the tissue against the inner walls of said cavity.

2. The bone wedge according to claim 1, wherein at least a tissue opening is formed at the distal end.

3. The bone wedge according to claim 2, further comprising a longitudinal groove, wherein said groove is formed on at least one of the two opposed sidewalls from the proximal end to the opening.

4. The bone wedge according to claim 2, wherein
a) the tissue opening has an elliptical cross-section, and/or
b) the cross-sectional area of said opening is preferably equal to or larger than the maximum cross-sectional area of the elongate body perpendicular to the longitudinal axis (A).

5. The bone wedge according to claim 1, wherein at least two or more teeth are formed on at least one of the two opposed sidewalls.

6. The bone wedge according to claim 1, wherein the maximum cross-section of the elongate body perpendicular to the longitudinal axis (A) is located at the proximal part of the body, preferably within the proximal third of the body, more preferably at that part of the body where the teeth are formed.

7. The bone wedge according to claim 5, wherein the teeth are formed on the two opposed sidewalls.

8. The bone wedge according to claim 5, wherein at least one of the teeth is inclined with regard to a plane perpendicular to the local surface outline of the wedge, wherein the angle of inclination ranges preferably between 1° and 89°, more preferably between 30° and 60°.

9. The bone wedge according to claim 8, wherein at least one of the teeth of each of the two opposed sidewalls is inclined and the direction of inclination of the teeth on the two opposed sidewalls is contrariwise.

10. The bone wedge according to claim 5, wherein the teeth of one sidewall of the two opposed sidewalls are shifted along the longitudinal axis (A) with respect to the teeth of the other sidewall the two opposed sidewalls.

11. The bone wedge according to claim 5, wherein a plurality of teeth of a sidewall of the two opposed sidewalls are shifted along the longitudinal axis (A) with respect to an adjacent tooth on said sidewall.

12. The bone wedge according to claim 5, wherein the teeth of the two sidewalls are arranged along aright-handed or left-handed helical curve/thread.

13. The bone wedge according to claim 1, wherein a bore, preferably with a bayonet fitting or with a female thread, for releasably connecting an insertion tool is formed at the proximal end, wherein said bore is preferably aligned along the longitudinal axis (A).

14. A kit for fixing tissue to bone comprising:
a bone wedge according to claim 1,
an insertion tool which is adapted for a releasable connection to the bone wedge;
a filament/tendon for extending a free end of the tissue to be fixed; and optionally
a special tool for creating a tapered or wedge-shaped bone cavity by impacting cancellous bone.

15. A method for fixing tissue to bone by means of a bone wedge, preferably a bone wedge according to claim. 1, the method comprising the steps:
a) forming a tapered cavity in the bone having a smaller diameter at its portion proximal to the bone surface than at its distal portion;
b) providing the bone wedge and connecting an insertion tool at the proximal end of the bone wedge;
c) extending a free end of the tissue to be fixed with a filament which has a smaller cross-section than said tissue;
d) inserting the filament into the opening of the bone wedge;
e) inserting the bone wedge together with said filament into the cavity such that the filament is guided from the proximal end of the body along the first sidewall through the opening at the distal end and back from the distal end to the proximal end;
f) pulling at the filament while pushing the insertion tool and thereby inserting the tissue into the cavity from the proximal end of the body along the teeth the first sidewall through the opening and reverse from the distal end of the body along the teeth the second sidewall towards the proximal end of said bone wedge and finally pulling the tissue out of the cavity again.

16. The method according to claim 15, wherein the cavity in the bone comprises a depth which is between 1.5 and 2 times the depth of the longitudinal extension of the bone wedge.
